(19) United States
(12) Patent Application Publication
(10) Pub. No.: US 2019/0070223 A1
(43) Pub. Date: Mar. 7, 2019

(54) Selection Method Of Highly Active Stem Cell For Treatment Of Intraventricular Hemorrhage In Preterm Infant

(71) Applicant: SAMSUNG LIFE PUBLIC WELFARE FOUNDATION, Seoul (KR)

(72) Inventors: Yun Sil CHANG, Seoul (KR); Won Soon PARK, Seoul (KR); So Yoon AHN, Seoul (KR); Dong Kyung SUNG, Seoul (KR)

(73) Assignee: SAMSUNG LIFE PUBLIC WELFARE FOUNDATION, Seoul (KR)

(21) Appl. No.: 16/086,781

(22) PCT Filed: Mar. 30, 2017
(86) PCT No.: PCT/KR2017/003469

§ 371 Date: Sep. 20, 2018

(30) Foreign Application Priority Data
Apr. 27, 2016 (KR) 10-2016-0051654
Mar. 29, 2017 (KR) 10-2017-0040158

(51) Int. Cl. A61K 35/28; C12N 5/0775; A61P 9/10; G01N 33/50; A61K 38/48; A61K 38/18; C12N 5/077; C12N 5/0735; C12N 5/073; A61K 35/50

(52) U.S. Cl. CPC A61K 35/28; C12N 5/0665; A61P 9/10; G01N 33/50; A61K 38/4833; C12Y 304/21005; C12N 5/0669; C12N 5/0667; C12N 5/0606; C12N 5/0605; A61K 35/50; A61K 38/185

(57) ABSTRACT

The present invention relates to a selection method of a highly active stem cell for treatment of cerebrovascular diseases, comprising a step of measuring a level of a neurotrophic factor, and a highly active stem cell selected thereby. According to the present invention, BDNF secreted by mesenchymal stem cells mediate inhibitory effects on cell death, inflammation, astrogliosis, and posthemorrhagic hydrocephalus and plays a very important role in improving myelination after intraventricular hemorrhage. Hence, the method according to the present invention can be usefully applied to the treatment of various cerebrovascular diseases including intraventricular hemorrhage in preterm infants.

### TECHNICAL FIELD

The present invention relates to a method of selecting a highly active stem cell for the treatment of a cerebrovascular disease, which includes measuring a level of a neurotrophic factor, and a highly active stem cell selected using the method.

### BACKGROUND

Although therapeutic drugs for neonates have recently been developed, intraventricular hemorrhage (IVH) is a main disease that causes death and neurologic disorders in preterm infants and there are few effective therapies. Therefore, there is a very urgent need to develop a new therapy for IVH to improve the prognosis of this severe disease. Recently, the inventors of the present invention verified that, when human cord blood-derived mesenchymal stem cells are intraventricularly transplanted into newborn Sprague-Dawley rats, posthemorrhagic hydrocephalus and brain damage were significantly reduced after severe bleeding due to IVH.

In addition, it was confirmed that such a neuroprotective effect of the mesenchymal stem cells was proportionate to time, earlier cell transplantation exhibited a better effect, and local intraventricular administration exhibited a better therapeutic effect than intravenous administration. In addition, the inventors of the present invention have reported that transplantation of mesenchymal stem cells has a significant therapeutic effect on various diseases such as bronchopulmonary dysplasia, acute respiratory distress syndrome, and neonatal stroke via a paracrine anti-inflammatory effect and an apoptotic inhibitory effect, as compared to regeneration mechanisms.

Meanwhile, various growth factors, for example, brain-derived neurotrophic factor (BDNF), nerve growth factor (NGF), vascular endothelial growth factor (VEGF), an insulin-like growth factor (IGF), interleukins, and the like are known to improve the ability to restore brain damage after hypoxia and/or ischemia (Qu R, Li Y, Gao Q, Shen L, Zhang J, Liu Z, Chen X, Chopp M. Neurotrophic and growth factor gene expression profiling of mouse bone marrow stromal cells induced by ischemic brain extracts. Neuropathology 2007; 27(4): 355-63.). However, a particular paracrine factor, which exhibits a neuroprotective effect by transplantation of mesenchymal stem cells during severe intraventricular hemorrhage, and a mechanism thereof have not yet been discovered.

### SUMMARY

### Technical Problem

The present invention has been made in view of the above problems, and the inventors of the present invention identified a particular factor, which mediates a neuroprotective effect by transplantation of mesenchymal stem cells after severe intraventricular hemorrhage and an accurate mechanism thereof. That is, as a result of treating mesenchymal stem cells with thrombin and performing DNA and antibody microarray analysis to screen genes and proteins whose expression is increased in the cells, it was confirmed that the expression of the BDNF gene and protein is increased in common in the mesenchymal stem cells, and when BDNF was present or the expression thereof is inhibited by siRNA through in vitro and in vivo experiments, a neuroprotective effect exhibited by mesenchymal stem cells was confirmed, thus completing the present invention based on these findings.

Therefore, an object of the preset invention is to provide a method of selecting a highly active stem cell for the treatment of a cerebrovascular disease, which includes measuring a level of a neurotrophic factor.

In addition, another object of the present invention is to provide a method of determining the activity of stem cells in treating a cerebrovascular disease in vitro, which includes measuring an expression level of a neurotrophic factor gene or protein of the stem cells and comparing the measured expression level with a reference value.

However, technical problems to be solved by the present invention are not limited to the above-described technical problems, and other unmentioned technical problems will become apparent from the following description to those of ordinary skill in the art.

### Technical Solution

According to an aspect of the present invention, there is provided a method of selecting a highly active stem cell for the treatment of a cerebrovascular disease, which includes measuring a level of a neurotrophic factor.

In one embodiment of the present invention, the method includes the following processes:

(a) culturing stem cells and treating the stem cells with thrombin;

(b) measuring a concentration of a neurotrophic factor in a culture solution of process (a); and

(c) identifying neuron protective activity of the stem cells based on the measured concentration.

In another embodiment of the present invention, the identifying of the neuron protective activity is performed such that, in a case in which the measured concentration of neurotrophic factor is 20 μg/ml or more, the case is determined as highly active.

In another embodiment of the present invention, the identifying of the neuron protective activity is performed such that, in a case in which the measured concentration of neurotrophic factor is 40 μg/ml or more, the case is determined as highly active.

In another embodiment of the present invention, thrombin of process (a) is included in a medium at a concentration of 1 unit/ml to 1,000 units/ml.

In another embodiment of the present invention, the neurotrophic factor is brain-derived neurotrophic factor (BDNF).

In another embodiment of the present invention, the cerebrovascular disease comprises neonatal intraventricular hemorrhage (IVH).

In another embodiment of the present invention, the highly active is neuron protective ability.

In another embodiment of the present invention, the stem cells are stem cells selected from the group consisting of mesenchymal stem cells, human tissue-derived mesenchymal stromal cells, human tissue-derived mesenchymal stem cells, multipotent stem cells, and amniotic epithelial cells.

In another embodiment of the present invention, the mesenchymal stem cells are derived from an umbilical cord, umbilical cord blood, bone marrow, fat, muscle, nerve, skin, an amniotic membrane, or a placenta.

The present invention also provides a method of determining the activity of stem cells in treating a cerebrovascular disease in vitro, which includes measuring an expression level of a neurotrophic factor gene or protein of the stem cells and comparing the measured expression level with a reference value.

The present invention also provides a highly active stem cell for treating a cerebrovascular disease, the highly active stem cell being selected using the method.

The present invention also provides a pharmaceutical composition for treating a cerebrovascular disease, which includes the highly active stem cell.

### Advantageous Effects of Invention

According to the present invention, BDNF, which is secreted by mesenchymal stem cells, plays a very important role in mediating an effect of inhibiting apoptosis, inflammation, astrogliosis, and the generation of posthemorrhagic hydrocephalus, and in enhancing myelination after intraventricular hemorrhage.

Therefore, the present invention provides a method of selecting a highly active stem cell for the treatment of a cerebrovascular disease, which includes measuring a level of a neurotrophic factor such as BDNF, and thus this method can be usefully used in treating various cerebrovascular diseases including neonatal intraventricular hemorrhage.

### BRIEF DESCRIPTION OF THE DRAWINGS

FIG. 1 illustrates analysis results showing a change in profiles of the expression of genes and proteins of mesenchymal stem cells after thrombin treatment, wherein FIG. 1A illustrates results of increased genes and proteins, FIG. 1B illustrates identification results of a cell survival rate of each of a plurality of groups, and FIG. 1C illustrates verification results of BDNF expression in mesenchymal stem cells.

FIG. 2 illustrates comparative analysis results showing the degree of ventricular enlargement in an intraventricular hemorrhage-induced control (IC), a group into which naive mesenchymal stem cells were transplanted (IM), a group into which mesenchymal stem cells transfected with scrambled siRNA were transplanted (IM-cont), and a group into which mesenchymal stem cells, in which BDNF expression was inhibited, were transplanted (IM-bdnf-kd), wherein FIG. 2A illustrates brain MRI results, and FIG. 2B illustrates calculation results of a volume ratio of total ventricles to whole brain.

FIG. 3 illustrates results of comparatively evaluating sensorimotor functions in the intraventricular hemorrhage-induced control (IC), the group into which naive mesenchymal stem cells were transplanted (IM), the group into which mesenchymal stem cells transfected with scrambled siRNA were transplanted (IM-cont), and the group into which mesenchymal stem cells, in which BDNF expression was inhibited, were transplanted (IM-bdnf-kd), wherein FIG. 3A illustrates negative geotaxis evaluation results, and FIG. 3B illustrates rotarod evaluation results.

FIG. 4 illustrates comparative analysis results of expression levels of human BDNF and Sprague-Dawley rat BDNF in the intraventricular hemorrhage-induced control (IC), the group into which naive mesenchymal stem cells were transplanted (IM), the group into which mesenchymal stem cells transfected with scrambled siRNA were transplanted (IM-cont), and the group into which mesenchymal stem cells, in which BDNF expression was inhibited, were transplanted (IM-bdnf-kd).

FIG. 5 illustrates comparative analysis results of myelination, apoptosis, and reactive gliosis in the intraventricular hemorrhage-induced control (IC), the group into which naive mesenchymal stem cells were transplanted (IM), the group into which mesenchymal stem cells transfected with scrambled siRNA were transplanted (IM-cont), and the group into which mesenchymal stem cells, in which BDNF expression was inhibited, were transplanted (IM-bdnf-kd), wherein FIG. 5A shows immunofluorescence staining microscope images, FIG. 5B shows western blotting results, FIG. 5C is a graph showing immunofluorescence staining results, and FIG. 5D is a graph showing western blotting results.

FIG. 6 illustrates comparative analysis results of inflammation of brain tissues around the ventricles in the intraventricular hemorrhage-induced control (IC), the group into which naïve mesenchymal stem cells were transplanted (IM), the group into which mesenchymal stem cells transfected with scrambled siRNA were transplanted (IM-cont), and the group into which mesenchymal stem cells, in which BDNF expression was inhibited, were transplanted (IM-bdnf-kd), wherein FIG. 6A shows immunofluorescence staining microscope images, FIG. 6B illustrates measurement results of the number of ED-1-positive cells, and FIG. 6C illustrates identification results of the levels of IL-1α, IL-1β, IL-6, and TNF-α.

FIG. 7 illustrates BDNF level analysis results according to stem cell source and lot.

### DETAILED DESCRIPTION

The present invention provides a method of selecting a highly active stem cell for the treatment of a cerebrovascular disease, which includes measuring a level of a neurotrophic factor. In the present invention, the method of selecting a highly active stem cell may include: (a) culturing stem cells and treating the stem cells with thrombin; (b) measuring a concentration of a neurotrophic factor in a culture solution of process (a); and (c) identifying neuron protective activity of the stem cells based on the measured concentration.

The term “cerebrovascular disease” as used herein refers to a neurologic deficit caused by normal blood supply disorders of the brain, and in the present invention, the cerebrovascular disease is preferably intraventricular hemorrhage (IVH).

The term “highly active” as used herein is intended to include significant excellence of the activity of stem cells or the therapeutic activity thereof against diseases, and preferably indicates the significant excellence of neuron protective ability.

In the present invention, in the selecting of the highly active stem cell, in a case in which the concentration of the neurotrophic factor is 20 pg/ml or more, more preferably 40 pg/ml or more, the case may be determined as highly active.

In the present invention, the neurotrophic factor is preferably brain-derived neurotrophic factor (BDNF).

In the present invention, the stem cells may be stem cells selected from the group consisting of mesenchymal stem cells, human tissue-derived mesenchymal stromal cells, human tissue-derived mesenchymal stem cells, multipotent stem cells, and amniotic epithelial cells. The mesenchymal stem cells may be derived from an umbilical cord, umbilical cord blood, bone marrow, fat, muscle, nerve, skin, an amniotic membrane, or a placenta, but the present invention is not limited thereto.

Mesenchymal stem cell (MSC) transplantation protects brain damage due to severe intraventricular hemorrhage (IVH) via a paracrine mechanism, rather than regeneration mechanisms in neonates. However, how the paracrine mechanism acts has not yet been clearly found.

As a result of identifying a particular factor, which mediates a neuroprotective effect by mesenchymal stem cell transplantation after severe intraventricular hemorrhage, and an accurate mechanism thereof, the inventors of the present invention verified that the expression of the BDNF gene and the expression of the BDNF protein were increased in common in mesenchymal stem cells, and verified a difference in neuroprotective effect exhibited by mesenchymal stem cells when BDNF was present or the expression thereof was inhibited using siRNA, through in vitro and in vivo experiments.

Specifically, in one embodiment of the present invention, as a result of treating fibroblasts and mesenchymal stem cells with thrombin and then performing DNA and antibody microarrays thereon, it was confirmed that the expression of brain-derived neurotrophic factor (BDNF) was significantly increased in the mesenchymal stem cells compared to the fibroblasts (see Example 2).

In another embodiment of the present invention, human BDNF-specific siRNA was transfected into mesenchymal stem cells to inhibit the expression of BDNF in the cells. To verify a therapeutic effect of mesenchymal stem cells in a state in which BDNF was present or the expression of BDNF was inhibited, an in vitro experiment was carried out using thrombin-treated neurons of Sprague-Dawley rats, and an in vivo experiment was also performed using newborn Sprague-Dawley (SD) rats. 200 μl of blood was administered into SD rats on day 4 after birth to induce intraventricular hemorrhage, and 1×10⁵ mesenchymal stem cells were transplanted into the ventricles of the SD rats on day 6 after birth.

As a result of the in vitro experiment, it was confirmed that, when the expression of BDNF was inhibited using BDNF siRNA, an apoptosis inhibitory effect of the mesenchymal stem cells on thrombin-induced neuronal death was not exhibited. Also, as a result of the in vivo experiment, it was confirmed that, when the expression of BDNF was inhibited, therapeutic effects on brain damage due to intraventricular hemorrhage, such as alleviation of posthemorrhagic hydrocephalus, mitigation of damage to the ability to perform behavioral tests, a reduction in astrogliosis increase, inhibition of an increase in the number of TUNEL-positive and ED-1-positive stained cells and in inflammatory cytokines, and an increase in expression reduction of myelin proteins, were not exhibited (see Examples 3 to 7).

In another embodiment of the present invention, as a result of examining a difference in BDNF level according to each source and a difference in cell survival rate according thereto, it was confirmed that the degree of BDNF secretion was exhibited in different forms according to each source and lot, and the cell survival rate was increased according to the secreted BDNF level (see Example 8).

The results of the examples suggest that BDNF secreted by transplanted mesenchymal stem cells is a very important paracrine factor exhibiting a neuroprotective effect, and BDNF may be used as a biomarker for selecting mesenchymal stem cells having the most excellent neuroprotective effect on intraventricular hemorrhage.

Therefore, according to another embodiment of the present invention, there is provided a method of determining the activity of stem cells in treating a cerebrovascular disease in vitro, which includes measuring an expression level of a neurotrophic factor gene or protein of the stem cells and comparing the measured expression level with a reference value.

According to another embodiment of the present invention, there is provided a highly active stem cell selected by the method and a pharmaceutical composition for treating a cerebrovascular disease, which includes the stem cell.

According to another embodiment of the present invention, there is provided a method of treating a cerebrovascular disease by transplanting the highly active stem cells selected by the method into an individual.

The term “individual” as used herein refers to a subject with diseases requiring treatment and, more particularly, includes mammals such as humans or non-human primates, e.g., mice, rats, dogs, cats, horses, cows, and the like.

Hereinafter, examples will be provided to aid in understanding the present invention. However, the following examples are provided only to more easily understand the present invention and are not intended to limit the scope of the present invention.

### EXAMPLES

### Example 1: Experimental Method

1-1. Cell Preparation

Umbilical cord blood-derived mesenchymal stem cells were obtained from Medipost Co., Ltd., Seoul, Korea, and an MRC-5 (Korean Cell Line Bank No. 10171) cell line, which consists of human fibroblasts, was purchased from Korean CellLine Bank, Seoul, Korea.

1-2. DNA and Antibody Microarray Analyses

To search for factors exhibiting neuroprotective effects at transcriptional and translational levels, gene and protein expression changes in mesenchymal stem cells were examined. Mesenchymal stem cells and MRC-5 cells, which are fibroblasts, were treated with thrombin for 6 hours and then analysis was performed thereon, RNA analysis was performed using the Illumina HumanHT-12 v4 Expression BeadChip, and protein analysis was performed using antibody array chips.

1-3. BDNF siRNA Transfection

Each of BDNF siRNA (sc-42121) and scrambled siRNA (sc-37007) was purchased from Santa Cruz Biotechnology, and each siRNA was transfected into mesenchymal stem cells using oligofectamine (Invitrogen, Carlsbad, Calif., USA) in accordance with the manufacturer's protocol. All analyses or mesenchymal stem cell transplantation was performed after each siRNA was transfected into mesenchymal stem cells for 24 hours. To identify whether the expression of BDNF was inhibited by BDNF siRNA after transfection, culture media of the mesenchymal stem cells were recovered and BDNF expression levels were measured. As a result, 24 hours after transfection, BDNF expression was decreased up to 27% of that of non-transfected mesenchymal stem cells.

1-4. Thrombin Exposure and In Vitro Cell Culture

For cell culture, brain neurons isolated from E18.5 embryonic mice were primarily cultured, and 5×10³ cells/well of neurons were seeded on a 96-well plate, and then cultured in 100 μl of a neurobasal medium containing a B-27 supplement (GIBCO, Gaithersburg, Md., USA) per each well at 37° C. for 24 hours.

Subsequently, to induce nerve damage due to hemorrhage in vitro, the cells were treated with 40 U of thrombin (Reyon Pharm. Co. Ltd, Seoul, South Korea), and the thrombin-treated neurons were cultured alone in a complete medium, or co-cultured with non-transfected umbilical cord blood-derived mesenchymal stem cells (1×10³), scrambled siRNA-transfected mesenchymal stem cells, or BDNF siRNA-transfected mesenchymal stem cells by seeding on an upper chamber for 24 hours. Thereafter, BDNF-blocking antibodies (Abcam, Cambridge, Mass., USA) or control immunoglobulin were added to the wells in which the thrombin-treated neurons and the non-transfected mesenchymal stem cells were co-cultured. In addition, a low (100 pg/ml) or high (1 ng/ml) concentration of recombinant human BDNF (R&D Systems, Minneapolis, Minn., USA) was added to the neurons that had been co-cultured with the BDNF siRNA-transfected mesenchymal stem cells.

Meanwhile, to measure cell viability, a colorimetric 3-(4,5-dimethylthiazol-2-yl)-2,5-diphenyl tetrazolium (MTT) assay (Dojindo Molecular Technologies Inc., Gaithersburg, Md., USA) was performed according to the manufacturer's protocol. Relative cell viability was corrected such that result values in the case of absence of cells were set at 0% and results values of non-treated cells were set at 100%. In addition, expression levels of genes and proteins between mesenchymal stem cells and fibroblasts were compared and analyzed through DNA and antibody microarrays.

1-5. Animal Model

All experimental protocols were carried out after being approved by the Research Animal Laboratory Committee of Samsung Biomedical Research Institute (Korea). Newborn Sprague-Dawley (SD) rats were used as an experimental animal, and the experiments were performed from day 4 (P4) to day 32 (P32) after birth. To induce intraventricular hemorrhage in the P4 SD rats, the SD rats were anesthetized with an anesthetic prepared by mixing halothane and nitrous oxide: oxygen in a ratio of 2:1, and then 200 μl of blood was collected from mother SD rats and 100 μl of blood for each case was injected into the ventricles on opposite sides of the brain. Intraventricular hemorrhage was induced and brain magnetic resonance imaging (MRI) was performed to identify the degree of intraventricular hemorrhage in SD rats on P5, which is one day after the intraventricular hemorrhage, and the cases in which intraventricular hemorrhage was barely induced or was not visually observed were excluded from the analyses.

Afterwards, on P 6, the SD rats, in which intraventricular hemorrhage had been induced, were randomly selected and divided into 5 groups as follows: a normal control (NC, n=16), an intraventricular hemorrhage-induced control (IC, n=18), a group into which naïve mesenchymal stem cells were transplanted (IM, n=16), a group into which scrambled siRNA-transfected mesenchymal stem cells were transplanted (IM-cont, n=17), and a group into which BDNF siRNA-transfected mesenchymal stem cells were transplanted (IM-kd, n=17). While the experiments were carried out, all SD rates, which belonged to the normal control (NC=16) in which intraventricular hemorrhage was not induced, survived up to P32, which was the last day of the experiments, whereas some SD rats in each group, in which intraventricular hemorrhage was induced, died, and thus these SD rats were excluded and then experiments were carried out (IC=4, IM=1, IM-cont=2, and IM-kd=5). For mesenchymal stem cell transplantation, 1×10⁵ naïve mesenchymal stem cells, 1×10⁵ scrambled siRNA-transfected mesenchymal stem cells, or 1×10⁵ BDNF siRNA-transfected mesenchymal stem cells were administered into the ventricle on the right side of the brain of each of the SD rats belonging to the IM, IM-cont, and IM-kd groups along with 10 μl of general saline. The same volume of saline was administered to the SD rats belonging to the IC group in which mesenchymal stem cells were not transplanted. Then, brain MRI image results of each group on P11 and P32 were obtained, and all the groups of SD rats on P32 were euthanized and then brain tissue samples were collected therefrom.

Meanwhile, as behavioral evaluation for evaluating sensorimotor nerves of the SD rats, negative geotaxis evaluation and rotarod evaluation were performed according to a conventionally known method (Ahn S Y, Chang Y S, Sung D K, Sung S I, Yoo H S, Lee J H, Oh W I, Park W S. Mesenchymal stem cells prevent hydrocephalus after severe intraventricular hemorrhage. Stroke 2013; 44(2):497-504; Ahn S Y, Chang Y S, Sung D K, Sung S I, Yoo H S, Im G H, Choi S J, Park W S. Optimal route for mesenchymal stem cells transplantation after severe intraventricular hemorrhage in newborn rats. PLoS One 2015; 10(7):e0132919).

1-6. TUNEL Assay

To analyze whether or not apoptosis occurs, a TUNEL assay was performed using white matter tissues around the ventricles according to a conventionally known method (Ahn S Y, Chang Y S, Sung D K, Sung S I, Yoo H S, Lee J H, Oh W I, Park W S. Mesenchymal stem cells prevent hydrocephalus after severe intraventricular hemorrhage. Stroke 2013; 44(2):497-504; Ahn S Y, Chang Y S, Sung D K, Sung S I, Yoo H S, Im G H, Choi S J, Park W S. Optimal route for mesenchymal stem cells transplantation after severe intraventricular hemorrhage in newborn rats. PLoS One 2015; 10(7):e0132919).

1-7. Immunohistochemistry Staining

To analyze the degrees of reactive gliosis, reactive microglia, and myelination, expression levels of glial fibrillary acidic protein (GFAP), ED-1, and myelin basic protein (MBP), which are neuron-specific, in tissues around the ventricles were evaluated via immunohistochemistry staining.

More specifically, deparaffinized brain coronal sections having a thickness of 4 μm were cultured with primary antibodies, and the primary antibodies used are as follows: GFAP (rabbit polyclonal; 1:1,000 dilution, Dako, Glostrup, Denmark), MBP (rabbit polyclonal; 1:1,000 dilution; Abcam), and ED-1 (mouse monoclonal; 1:100 dilution; Millipore).

Subsequently, three coronal sections (+0.95 mm to −0.11 mm/bregma) of each brain were stained, and three random fields that do not overlap with one another in a periventricular region, including the corpus callosum and the caudate nucleus, of each section were evaluated. The immunofluorescence intensity of GFAP or MBP staining of each randomly selected field was measured using ImageJ software [National Institutes of Health (NIH), Bethesda, Md., USA], and the number of ED-1+ cells was also counted in the randomly selected fields.

1-8. Enzyme-Linked Immunosorbent Assay (ELISA)

To measure the expression levels of inflammatory cytokines, i.e., IL-1α, IL-1β, IL-6, and TNF-α, an ELISA was performed using a homogeneous suspension of periventricular tissue.

More specifically, frozen samples of brain tissue obtained from the periventricular region were homogenized and centrifuged at 8,000×g and 4° C. for 20 minutes. The amounts of proteins in the supernatant were measured using the Bradford method using bovine serum albumin (BSA, Sigma-Aldrich, St. Louis, Mo., USA) as a standard solution. The levels of inflammatory cytokines including IL-1α, IL-1β, IL-6, and TNF-α were measured using a homogeneous suspension of periventricular tissue and a Milliplex MAP ELISA kit. Human- and SD rat-specific BDNF was measured using an ELISA kit (Quantikine ELISA Kit, R & D Systems) according to the manufacturer's protocol.

1-9. Statistical Analyses

Sample size measurement was based on ventricular volume differences on P32 according to power 0.8 and Type I error probability 0.05, which were previous study results. Experimental data was expressed as mean±standard deviation.

Microarray data was analyzed using the ANOVA (GeneSpring, Agilent Technologies, Santa Clara, Calif., USA) program with Benjamini-Hochberg correction for multiple comparisons. For continuous variability, statistical comparison between the groups was performed by one-way ANOVA and Tukey's post hoc analyses. To analyze changes over time, Tukey's post hoc comparison was performed using a univariate general linear model to perform repeated measurements. All data was analyzed using SPSS version 18.0 (IBM, Chicago, Ill., USA), and the case of P<0.05 was determined as statistically significant.

### Example 2: Analysis of Profile Changes of Gene and Protein Expression of Mesenchymal Stem Cells after Thrombin Treatment

To identify changes in the expression of genes and proteins associated with the neuroprotective effect of mesenchymal stem cells after being treated with thrombin, DNA and antibody microarray analyses were performed.

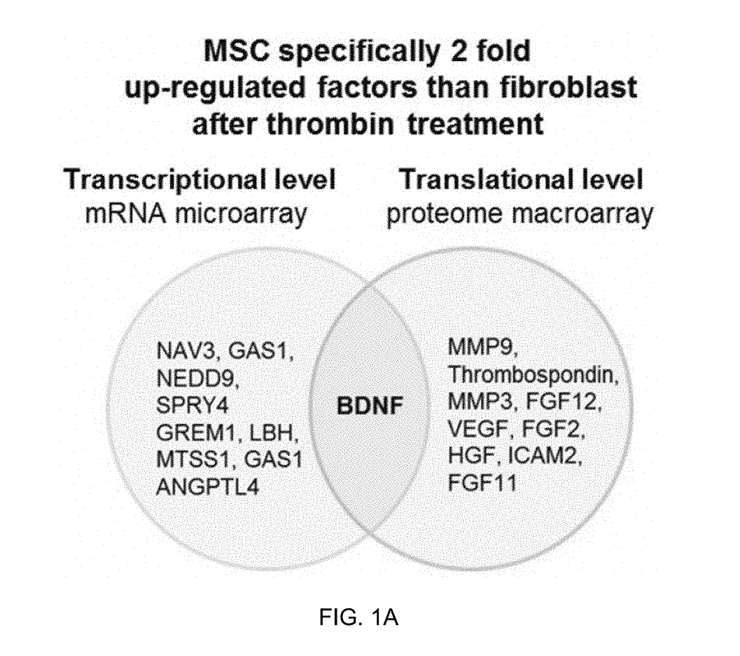

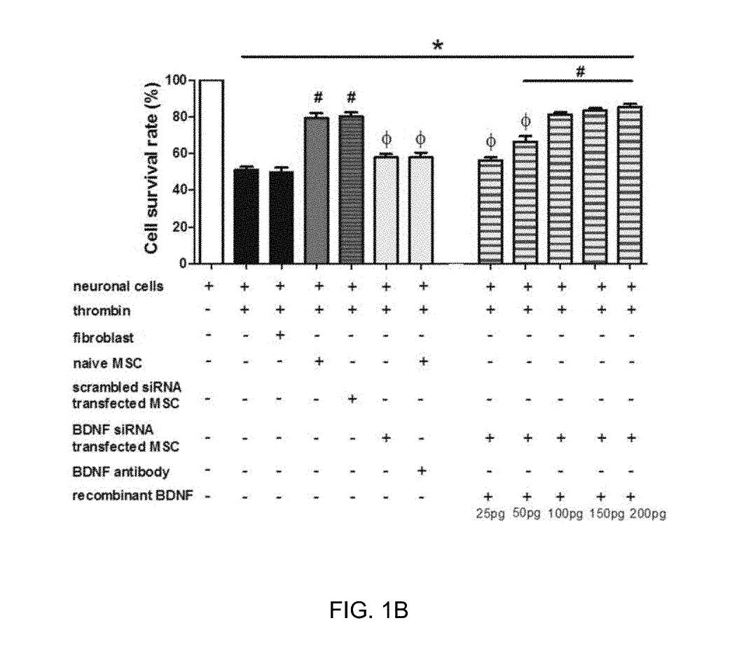

As a result, as illustrated in FIG. 1A, it was confirmed that, in the mesenchymal stem cells, the expression levels of 46 genes were significantly increased as a result of the DNA microarray analysis, and the expression levels of 12 proteins were significantly increased as a result of the antibody microarray analysis, as compared to human fibroblasts. As a result of analyzing the top 10 genes and the top 10 proteins, which exhibited the most significantly increased expression levels, among the genes and proteins, it was confirmed that the expression of BDNF was increased at both gene and protein levels.

### Example 3: In Vitro Verification of Neuroprotective Effect of Mesenchymal Stem Cells

To verify a neuroprotective effect of mesenchymal stem cells in vitro, neurons isolated from SD rats and primarily cultured were treated with thrombin, and then a neuroprotective effect of mesenchymal stem cells in each rat was evaluated.

As a result, as illustrated in FIG. 1B, it was confirmed that cell viability was significantly reduced in a case in which neurons treated with 40 U of thrombin were cultured alone for 24 hours and a case in which the thrombin-treated neurons were co-cultured with fibroblasts. In contrast, neuron apoptosis was significantly decreased in a case in which the thrombin-treated neurons were co-cultured with human umbilical cord blood-derived mesenchymal stem cells. These results indicate that mesenchymal stem cells have a specific protective effect on neurons.

Furthermore, the neuron apoptosis inhibitory effect of mesenchymal stem cells was observed in a case in which the thrombin-treated neurons were co-cultured with naïve MSCs or scrambled siRNA-transfected mesenchymal stem cells, but was not observed in a case in which the thrombin-treated neurons were co-cultured with mesenchymal stem cells, in which the expression of BDNF was inhibited using BDNF siRNA. In addition, it was confirmed that the neuron protective effect of mesenchymal stem cells on thrombin-mediated neuronal death was not exhibited even in a case in which the thrombin-treated neurons were treated with BDNF-neutralizing antibodies. These results indicate that BDNF secreted by transplanted mesenchymal stem cells plays a vital role in mediating the neuron protective effect of mesenchymal stem cells.

In addition, in a case in which the thrombin-treated neurons were co-cultured with mesenchymal stem cells in which the expression of BDNF was inhibited, as a result of treatment with human recombinant BDNF at various concentrations (25 pg, 50 pg, 100 pg, 150 pg, and 200 pg), it was confirmed that, when treated at concentrations of 100 pg or more, the neuroprotective effect of mesenchymal stem cells was recovered, and these concentration-dependent results were seen as results corresponding to the concentration (125±16 pg/ml) of BDNF expressed in naïve mesenchymal stem cells (see FIG. 1C).

### Example 4: Brain MRI Analysis

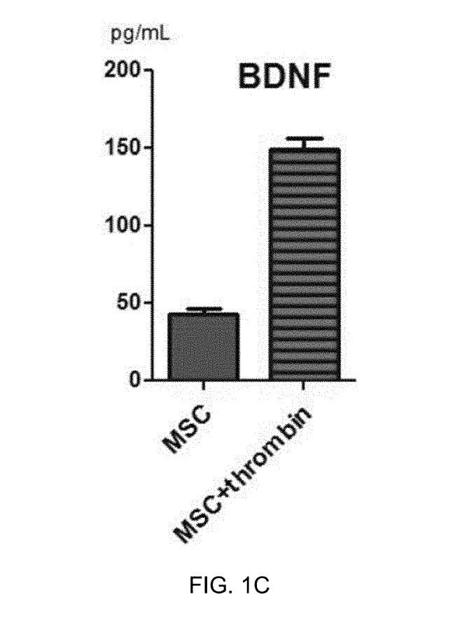

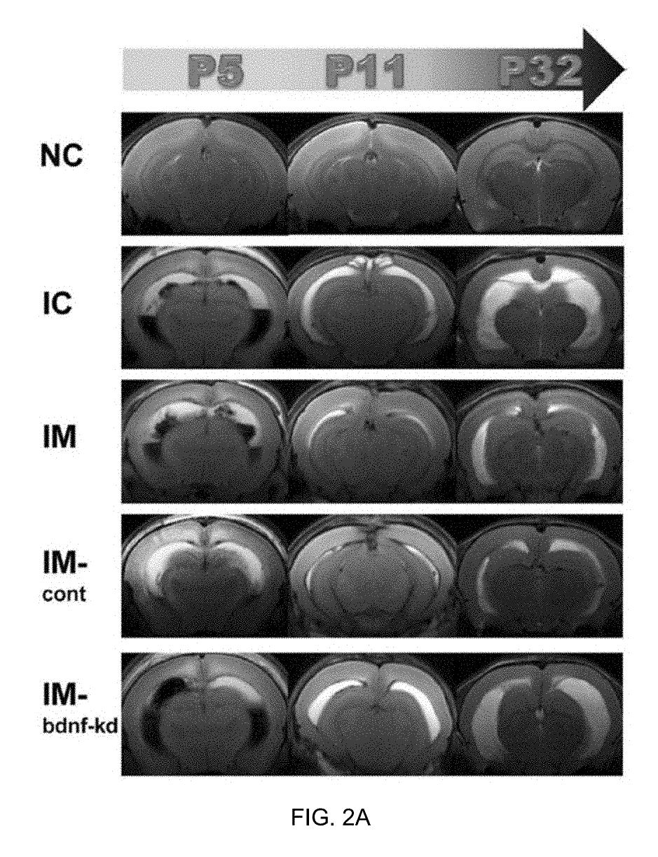

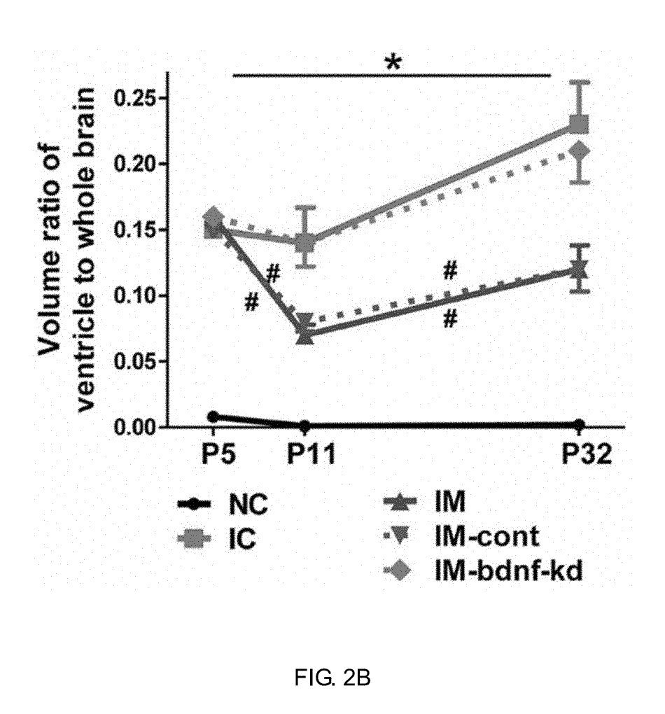

Brain MRI was performed on each group of SD rats on day 1, day 7, and day 28 (P5, P11, and P32) after intraventricular hemorrhage induction, and the results thereof are shown in FIG. 2A.

As a result of measuring ventricular enlargement degrees by calculating a volume ratio of total ventricles to whole brain on P5, P11, and P32, as illustrated in FIG. 2B, all the groups did not show any significant difference on P5, whereas the intraventricular hemorrhage-induced control (IC) and the group into which mesenchymal stem cells, in which BDNF expression was inhibited, were transplanted (IM-bdnf-kd) exhibited significantly increased ventricular enlargement degrees on P11 and P32. In contrast, it was confirmed that the group into which naïve mesenchymal stem cells were transplanted (IM) or the group into which the scrambled siRNA-transfected mesenchymal stem cells were transplanted (IM-cont) exhibited significantly reduced ventricular enlargement degrees.

### Example 5: Sensorimotor Behavioral Evaluation and Analysis

To evaluate sensorimotor functions, negative geotaxis evaluation and rotarod evaluation were performed.

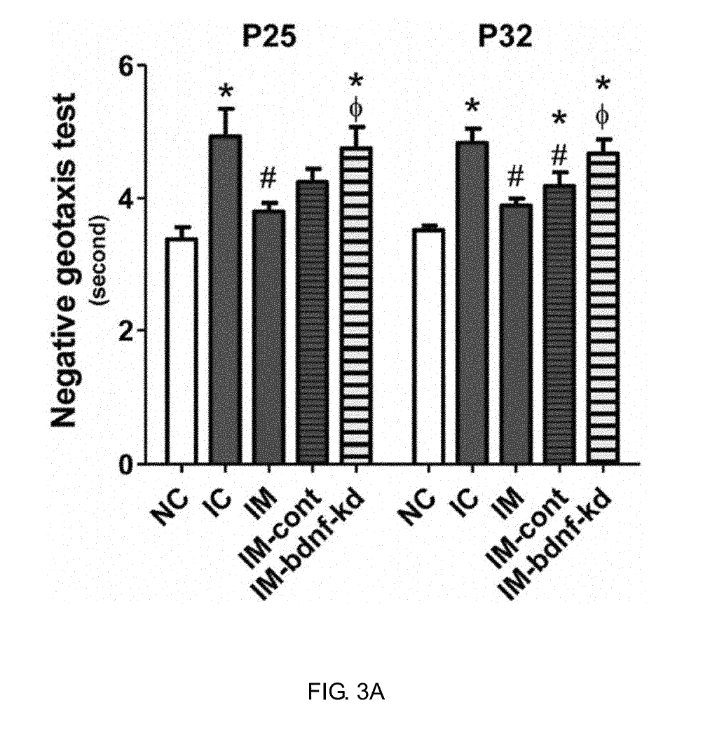

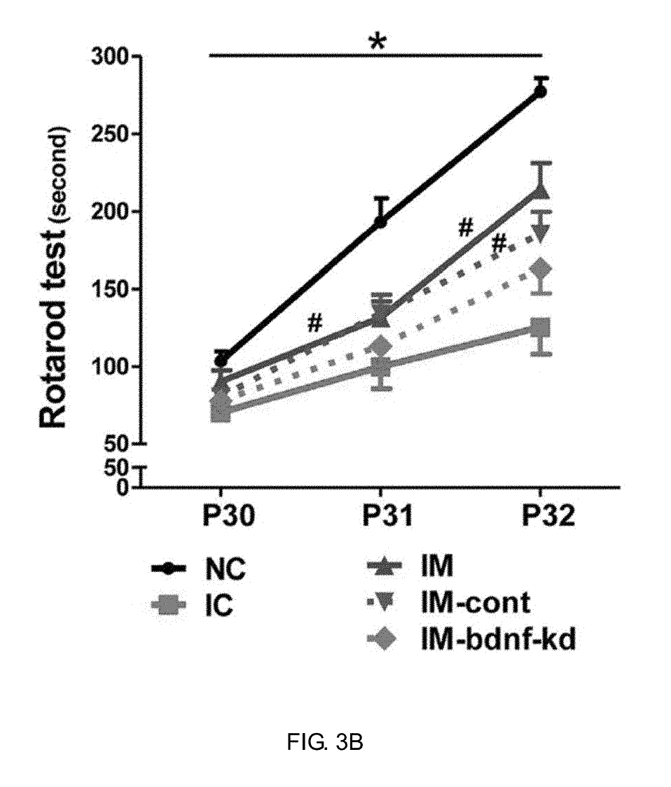

First, negative geotaxis evaluation was performed on P25 and P32, and for analysis, the head of each SD rat was placed on a slant board to face downward according to the conventionally known method, and the time taken for the head of each SD rat to face the back of the slant board was recorded. As a result, as illustrated in FIG. 3A, more severe motor function impairment was observed in the case of the intraventricular hemorrhage-induced control (IC) than in the normal control, whereas the impaired motor ability was significantly improved in the group into which naïve mesenchymal stem cells were transplanted (IM) or the group into which scrambled siRNA-transfected mesenchymal stem cells were transplanted (IM-cont). In addition, it was confirmed that a motor ability enhancement effect was not exhibited in the group into which mesenchymal stem cells, in which BDNF expression was inhibited, were transplanted (IM-bdnf-kd).

Next, rotarod evaluation was performed on each of P30, P31, and P32. As a result, as illustrated in FIG. 3B, when rotarod evaluation was first performed on P30, a significant difference between the groups was not exhibited, whereas the time taken for each rat to fall off of a rod was significantly increased on P31 and P32 due to a learning effect in the normal control (NC) and the time taken for each rat to fall off of the rod was measured to be significantly shorter on P31 and P32 in the intraventricular hemorrhage-induced control (IC) than in the normal control. However, it was confirmed that the impaired motor ability was significantly improved in the group into which naïve mesenchymal stem cells were transplanted (IM) or the group into which scrambled siRNA-transfected mesenchymal stem cells were transplanted (IM-cont), and the improvement effect was not exhibited in the group into which mesenchymal stem cells, in which BDNF expression was inhibited, were transplanted (IM-bdnf-kd).

### Example 6: Analyses of BDNF Expression Level, Myelination, Apoptosis, and Reactive Gliosis

6-1. BDNF Expression Level Analysis

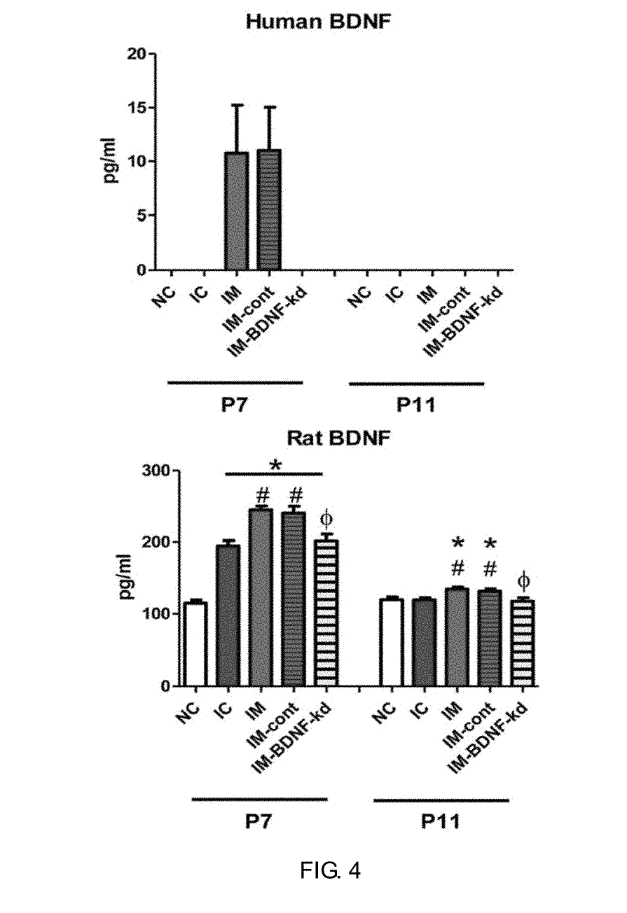

Human BDNF was measured in a brain homogenate suspension of each group of SD rats on P7, which is one day after mesenchymal stem cell transplantation. As a result, as illustrated in FIG. 4, human BDNF was measured in the group into which naïve mesenchymal stem cells were transplanted (IM) and the group into which scrambled siRNA-transfected mesenchymal stem cells were transplanted (IM-cont), whereas BDNF was not measured in the intraventricular hemorrhage-induced control (IC) and the group into which mesenchymal stem cells, in which BDNF expression was inhibited, were transplanted (IM-bdnf-kd).

In addition, as a result of measuring BDNF levels of the SD rats on P7, as illustrated in FIG. 4, BDNF levels of the SD rats were measured significantly higher in the group into which naïve mesenchymal stem cells were transplanted (IM) and the group into which scrambled siRNA-transfected mesenchymal stem cells were transplanted (IM-cont) than in the intraventricular hemorrhage-induced control (IC) and the group into which mesenchymal stem cells, in which BDNF expression was inhibited, were transplanted (IM-bdnf-kd), and also were higher than in the normal control. In addition, it was confirmed that human BDNF was not measured in any group on P11, which is five days after the mesenchymal stem cell transplantation, and the BDNF levels of the SD rats were significantly increased in the groups into which naïve mesenchymal stem cells and scrambled siRNA-transfected mesenchymal stem cells were respectively transplanted, as compared to the normal control.

6-2. Myelination Analysis

To evaluate the degree of myelination in periventricular tissue, immunostaining using MBP antibodies and western blotting were performed.

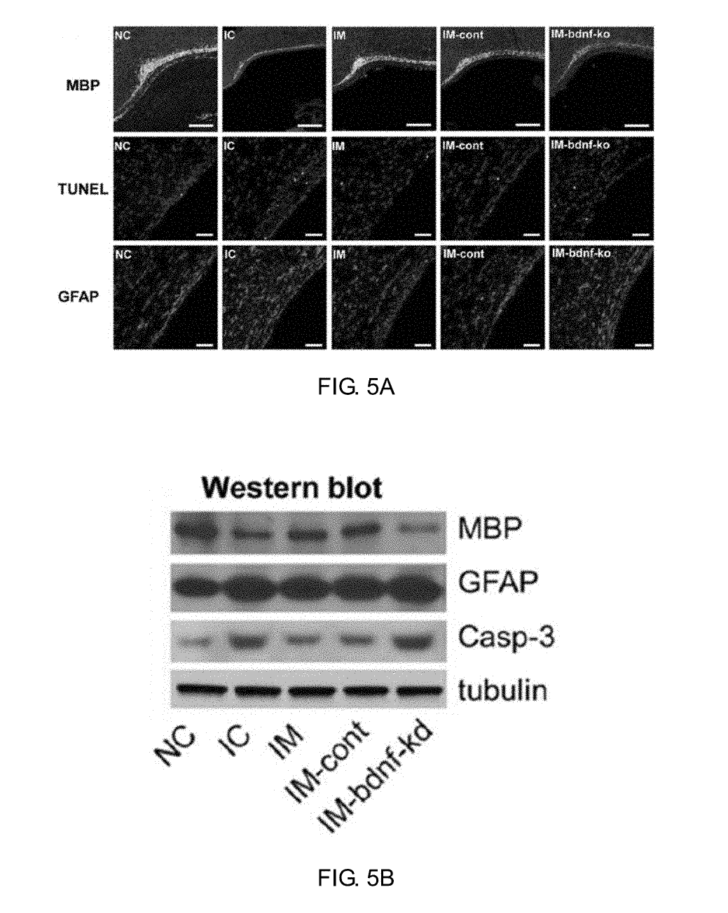

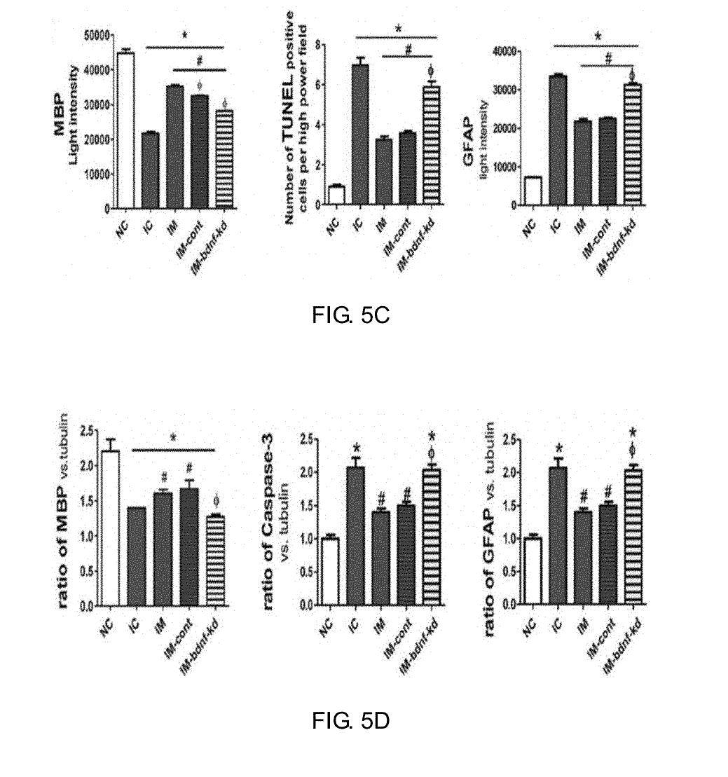

As a result, as illustrated in FIGS. 5A and 5B, the expression of MBP protein was significantly reduced in the intraventricular hemorrhage-induced control (IC) as compared to the normal control (NC). However, it was confirmed that such myelination impairment was considerably alleviated in the groups into which naïve mesenchymal stem cells and scrambled siRNA-transfected mesenchymal stem cells were respectively transplanted (IM and IM-cont), and thus the expression level of MBP was increased. In contrast, it was confirmed that the alleviation effect was not exhibited in the group into which mesenchymal stem cells, in which BDNF expression was inhibited, were transplanted (IM-bdnf-kd).

6-3. Apoptosis Analysis

To identify the degree of apoptosis after intraventricular hemorrhage, the number of TUNEL-positive cells stained by a TUNEL reagent was measured by performing TUNEL analysis using periventricular tissue on P32, and an expression level of caspase-3 was identified via western blotting.

As a result, as illustrated in FIGS. 5A to 5C, the number of TUNEL-positive cells and the expression of caspase-3 were significantly increased in the intraventricular hemorrhage-induced control (IC). However, it was confirmed that the number of TUNEL-positive cells and the expression of caspase-3 were significantly reduced in the groups into which naïve mesenchymal stem cells and scrambled siRNA-transfected mesenchymal stem cells were respectively transplanted (IM and IM-cont). In contrast, it was confirmed that the apoptosis inhibitory effect was not exhibited in the group into which mesenchymal stem cells, in which BDNF expression was inhibited, were transplanted (IM-bdnf-kd).

6-4. Reactive Gliosis Analysis

Reactive gliosis in periventricular tissue was evaluated by observing GFAP-stained cells via immunohistochemistry staining and measuring the expression level of GFAP protein by western blotting.

As a result, as illustrated in FIGS. 5A to 5C, it was confirmed that the degree of GFAP staining and protein expression were increased in the intraventricular hemorrhage-induced control (IC), whereas the degree of GFAP staining and protein expression were reduced in the groups into which naïve mesenchymal stem cells and scrambled siRNA-transfected mesenchymal stem cells were respectively transplanted (IM and IM-cont). In contrast, it was confirmed that the above-described reduction effects were not exhibited in the group into which mesenchymal stem cells, in which BDNF expression was inhibited, were transplanted (IM-bdnf-kd).

### Example 7: Inflammation Analysis of Periventricular Tissue

To verify whether the transplanted mesenchymal stem cells alleviated brain inflammation induced by intraventricular hemorrhage, the levels of inflammatory cytokines, i.e., IL-1α, IL-1β, IL-6, and TNF-α, from a homogenous suspension of periventricular tissue were measured on P32, and the number of ED-1-positive cells in brain coronal sections was measured for analysis.

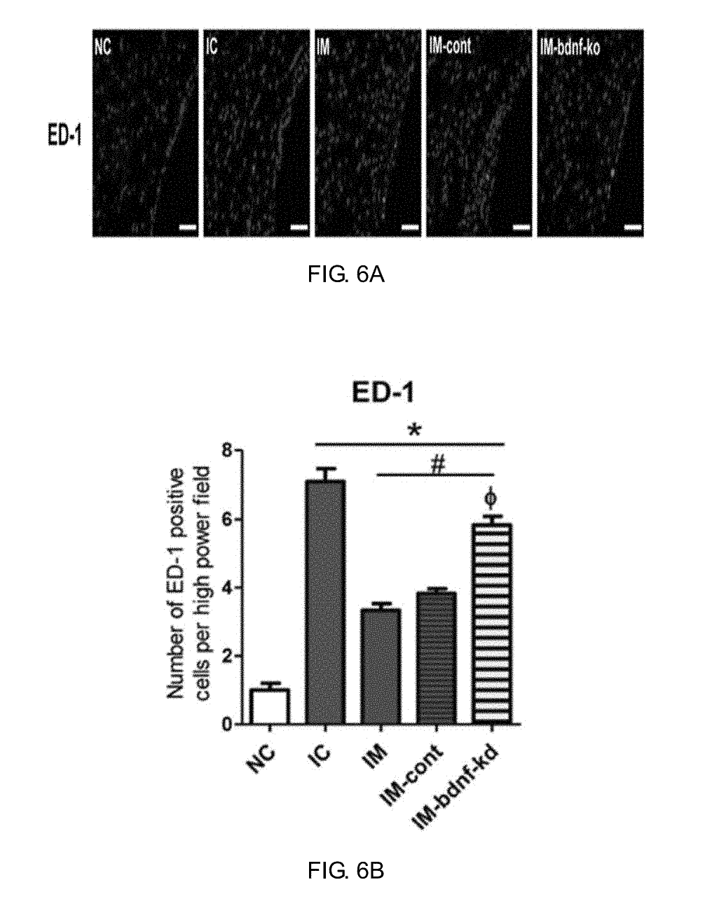

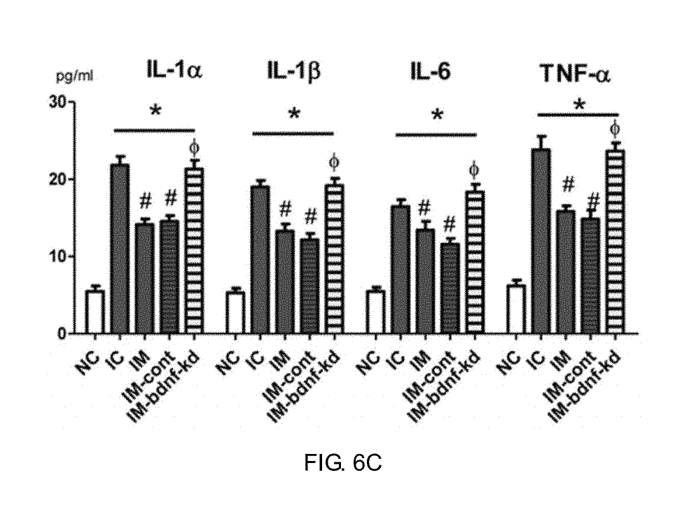

As a result, as illustrated in FIGS. 6A to 6C, it was confirmed that the levels of ED-1-positive cells and inflammatory cytokines in brain periventricular tissue were significantly increased in the intraventricular hemorrhage-induced control (IC) as compared to the normal control. In contrast, it was confirmed that the levels of ED-1-positive cells and inflammatory cytokines were decreased in the groups into which naïve mesenchymal stem cells and scrambled siRNA-transfected mesenchymal stem cells were respectively transplanted (IM and IM-cont), and the decreased effects were not exhibited in the group into which mesenchymal stem cells, in which BDNF expression was inhibited, were transplanted (IM-bdnf-kd).

### Example 8: Prediction/Selection of Highly Active Stem Cell by BDNF Level Analysis

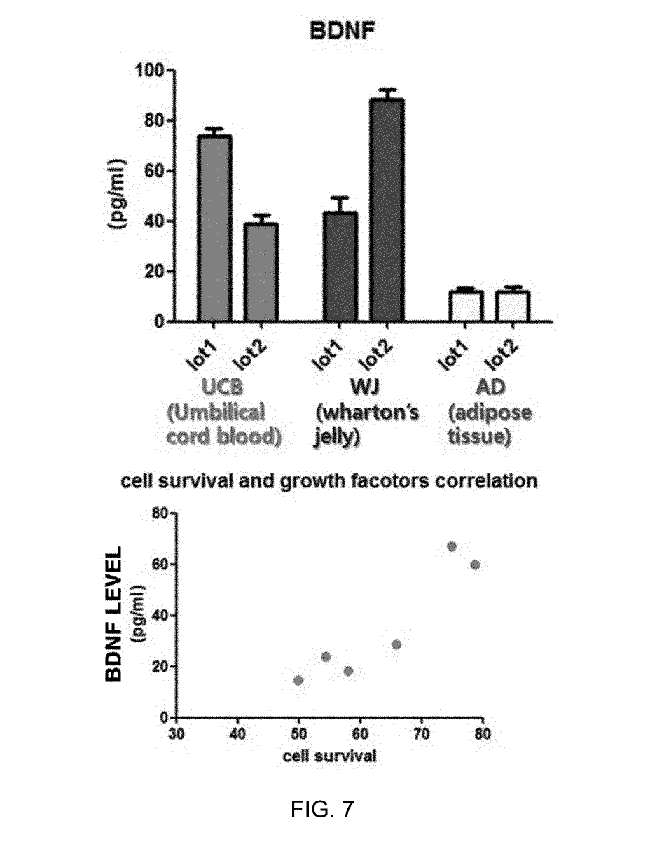

As a result of culturing two lots of each of umbilical cord blood-derived mesenchymal stem cells (UCB), umbilical cord-derived mesenchymal stem cells (WJ), and adipose-derived mesenchymal stem cells (AD), measuring BDNF levels of culture solutions thereof by ELISA, and comparing the BDNF levels with each other, as illustrated in the upper side of FIG. 7, it was confirmed that the degrees of BDNF secretion were different according to each source and each lot.

In addition, neurons obtained from the embryo brain of each mouse after primary neuronal culture were treated with thrombin (40 units) for 4 hours to produce an in vitro intraventricular hemorrhage model. As a result of treating the model with mesenchymal stem cells exhibiting different BDNF expression levels according to lot, and then examining cell survival rates, as illustrated in the lower side of FIG. 7, it was confirmed that cell survival rates were increased according to the level of BDNF secreted according to each lot.

In particular, in a case in which the BDNF level is 20 pg/ml or more, preferably 40 pg/ml or more, and more preferably 60 pg/ml or more, the case may be determined as highly active in neuron protection.

These results indicate that a particular level of BDNF secreted by stem cells may be an index for predicting the neuron protective activity of the stem cells.

The foregoing description of the present invention is provided for illustrative purposes, and it will be understood by those of ordinary skill in the art to which the present invention pertains that the invention may be easily modified in many different forms without departing from the spirit or essential characteristics of the present invention. It is therefore to be understood that the above-described embodiments are illustrative in all aspects and not restrictive.

### INDUSTRIAL APPLICABILITY

The present invention provides a method of selecting a highly active stem cell for the treatment of a cerebrovascular disease, which includes measuring a level of a neurotrophic factor such as BDNF, and thus the method may be usefully used in treating various cerebrovascular diseases including neonatal intraventricular hemorrhage.

1. A method for selecting a highly active stem cell for the treatment of a cerebrovascular disease, the method comprising measuring a level of a neurotrophic factor.

2. The method according to claim 1, wherein the method comprises the following processes:
(a) culturing stem cells and treating the stem cells with thrombin;
(b) measuring a concentration of a neurotrophic factor in a culture solution of process (a); and
(c) identifying neuron protective activity of the stem cells based on the measured concentration.

3. The method according to claim 2, wherein the identifying of the neuron protective activity is performed such that, in a case in which the measured concentration of the neurotrophic factor is 20 pg/ml or more, the case is determined as highly active.

4. The method according to claim 3, wherein the identifying of the neuron protective activity is performed such that, in a case in which the measured concentration of the neurotrophic factor is 40 pg/ml or more, the case is determined as highly active.

5. The method according to claim 2, wherein thrombin of process (a) is included in a medium at a concentration of 1 unit/ml to 1,000 units/ml.

6. The method according to claim 1, wherein the neurotrophic factor is brain-derived neurotrophic factor (BDNF).

7. The method according to claim 1, wherein the cerebrovascular disease is neonatal intraventricular hemorrhage (IVH).

8. The method according to claim 1, wherein the highly active is neuron protective ability.

9. The method according to claim 1, wherein the stem cells are stem cells selected from the group consisting of mesenchymal stem cells, human tissue-derived mesenchymal stromal cells, human tissue-derived mesenchymal stem cells, multipotent stem cells, and amniotic epithelial cells.

10. The method according to claim 9, wherein the mesenchymal stem cells are derived from an umbilical cord, umbilical cord blood, bone marrow, fat, muscle, nerve, skin, an amniotic membrane, or a placenta.

11. A method for determining the activity of stem cells in treating a cerebrovascular disease in vitro, the method comprising measuring an expression level of a neurotrophic factor gene or protein of the stem cells and comparing the measured expression level with a reference value.

12. The method according to claim 11, wherein the stem cells are stem cells treated with thrombin.

13. The method according to claim 11, wherein the neurotrophic factor is brain-derived neurotrophic factor (BDNF).

14. The method according to claim 11, wherein the cerebrovascular disease is neonatal intraventricular hemorrhage (IVH).

15. The method according to claim 11, wherein the stem cells are stem cells selected from the group consisting of mesenchymal stem cells, human tissue-derived mesenchymal stromal cells, human tissue-derived mesenchymal stem cells, multipotent stem cells, and amniotic epithelial cells.

16. The method according to claim 15, wherein the mesenchymal stem cells are derived from an umbilical cord, umbilical cord blood, bone marrow, fat, muscle, nerve, skin, an amniotic membrane, or a placenta.

17. A highly active stem cell for the treatment of a cerebrovascular disease, the highly active stem cell being selected by the method of claim 1.

18. The highly active stem cell according to claim 17, wherein the cerebrovascular disease is neonatal intraventricular hemorrhage (IVH).

19. A method for treating a cerebrovascular disease, comprising:
administering to a subject in need thereof an effective amount of the highly active stem cell of claim 17.

20. The method according to claim 19, wherein the cerebrovascular disease is neonatal intraventricular hemorrhage (IVH).

21.-22. (canceled)
